(19) United States
(12) Patent Application Publication
(10) Pub. No.: US 2017/0259262 A1
(43) Pub. Date: Sep. 14, 2017

(54) LAMINAR FLUIDIC SEPARATION IN FLOWCELLS FOR MINIMAL REAGENT USAGE

(71) Applicants: Intelligent Bio-Systems, Inc., Waltham, MA (US); Qiagen GmbH, Hilden (DE)

(72) Inventors: Thomas Daniel Perroud, Lexington, MA (US); Michel Georges Perbost, Belmont, MA (US); Maximilian Focke, Dusseldorf (DE)

(21) Appl. No.: 15/447,664

(22) Filed: Mar. 2, 2017

Related U.S. Application Data
(60) Provisional application No. 62/305,636, filed on Mar. 9, 2016.

(51) Int. Cl. B01L 3/00; C12Q 1/68

(52) U.S. Cl. CPC B01L 3/502715; C12Q 1/6869; B01L 3/502776; B01L 2300/0887; B01L 2200/027; B01L 2200/0636; B01L 2200/16; B01L 2300/168; B01L 2300/0877; B01L 2300/0867; B01L 2200/0673

(57) ABSTRACT

A processing instrument flowcell having a flowcell channel with an upstream channel end, a downstream channel end, a longitudinal axis extending from the upstream channel end to the downstream channel end, and a first operative surface extending between the upstream channel end and the downstream channel end and configured to receive a first number of DNA templates. A first reagent inlet is fluidically connected to the upstream channel end at a location adjacent the first operative surface. A buffer inlet is fluidically connected to the upstream channel end at a location spaced from the first operative surface. An outlet fluidically connected to the downstream channel end. Also provided is a method for operating a flowcell channel under laminar flow conditions to maintain a first reagent adjacent a first operative surface and a buffer fluid spaced from the first operative surface. The flowcell channel may have multiple separate operative surfaces.

### CROSS REFERENCE TO RELATED APPLICATIONS

This application claims the benefit of U.S. Provisional Application No. 62/305,636, entitled LAMINAR FLUIDIC SEPARATION IN FLOWCELLS FOR MINIMAL REAGENT USAGE filed Mar. 9, 2016, the contents of which are incorporated herein by reference.

### BACKGROUND OF THE INVENTION

Field of the Invention

The present invention relates generally to instruments for performing sequencing-by-syntheses or other sequencing processes, and more particularly to flowcells used in such instruments.

Description of the Related Art

Deoxyribonucleic acid (“DNA”) sequencing instruments are used to determine DNA molecular sequences. Such instruments are useful for clinical studies, diagnostics, so-called “personalized medicine” (medical treatment tailored to an individual's genetic content or the like), and so on. Current instruments for performing DNA sequencing use a variety of technologies to analyze the base pairs that form the DNA sequence. For example, some instruments perform sequencing on single-stranded DNA molecule fragments (DNA templates) that are fixed in place inside a flowcell. The flowcell is essentially a small chamber in which the DNA templates are subjected to a series of nucleobase extension processes. Each successive extension is detected to determine the base pair sequence of each DNA template. The flowcell provides an environment to hold the DNA templates during the extension process, and also during the imaging process to read each extended base pair.

Many sequencing-by-synthesis instruments use an optical system such as a microscope to detect the nucleobase extensions, although non-optical systems are also known. A typical optical instrument uses visible chemical labels to determine the identity of each extended base pair. For example, each nucleobase (i.e., adenine, guanine, cytosine and thymine) that makes up the DNA molecule may be labeled with a unique fluorescent probe that is visible through the microscope. The label is read each time the DNA template is extended, and then the label is removed to make way for the next base pair extension.

In modern “next-generation” instruments, millions of DNA templates may be processed simultaneously in a single flowcell. The DNA templates may be randomly ordered within the flowcell, or ordered at specific predetermined locations. A variety of flowcell designs have been developed to hold the immobilized DNA templates, but they usually include certain common features. A typical flowcell includes a flow channel, an optically transparent cover that encloses the channel, and fluid inlets and outlets through which the appropriate reagents are passed to control the growth and extension of the DNA templates. Example of such flowcells are found in U.S. Pat. Nos. 8,481,259, 8,940,481 and 9,146,248 and U.S. Patent Application Publication Nos. 2009/0298131 and 2014/0267669, all of which are incorporated herein by reference.

The flowcell channel may be a so-called “microfluidic” channel, which is loosely defined as one that is less than about one millimeter in height. (As used herein, the “height” is the dimension perpendicular to a plane in which the DNA templates are immobilized.) Such flowcells typically are dimensioned to minimize the cross-sectional area and maximize the surface area-to-volume ratio. (As used herein, the “cross-sectional area” refers to the area in a plane perpendicular to the flow axis of the channel.) Increasing the surface area-to-volume ratio has the beneficial effect of increasing the exposure of the DNA templates to reagents passing through the flowcell. However, decreasing the cross-sectional area increases the amount of pressure differential required to flow reagents through the channel, and increases the likelihood that the channel will clog, and these factors may limit the ability to reduce the cross-sectional area. Typical flowcells are optimized, with respect to the height dimension, to balance these considerations and the maximum surface area-to-volume ratio without unduly decreasing the cross-sectional area.

The inventors have determined that there continues to be a need to advance the state of the art of flowcells for sequencing instruments and similar devices.

### SUMMARY

Non-limiting examples of embodiments are provided in the following summary.

In one exemplary aspect, there is provided a flowcell for a processing instrument. The flowcell has a flowcell channel having an upstream channel end, a downstream channel end, a longitudinal axis extending from the upstream channel end to the downstream channel end, and a first operative surface extending between the upstream channel end and the downstream channel end and configured to receive a first plurality of DNA templates. A first reagent inlet is fluidically connected to the upstream channel end at a location adjacent the first operative surface. A buffer inlet is fluidically connected to the upstream channel end at a location spaced from the first operative surface. An outlet is fluidically connected to the downstream channel end. The upstream channel end may have a predetermined height in a direction perpendicular to the longitudinal axis, and the first reagent inlet may have an exit portion, adjacent to the upstream channel end, that extends from a first point adjacent to the first operative surface to a second point that is spaced a predetermined distance from the first operative surface in the direction perpendicular the longitudinal axis. Such predetermined distance may be 0.4% to 50% of the predetermined height, 1.4% to 18% of the predetermined height, or 3.6% to 10.7% of the predetermined height. The predetermined height also may be 140 micrometers, and the exit portion may extend from a first point adjacent to the first operative surface to a second point that is spaced from the first operative surface in the direction perpendicular the longitudinal axis by about 0.5 micrometers to about 50 micrometers, about 2 micrometers to about 25 micrometers, or about 5 micrometers to about 15 micrometers. The first reagent inlet may include an exit portion, located immediately upstream of the upstream channel end, that is parallel to and coincident with the first operative surface. The buffer inlet may include an exit portion, located immediately upstream of the upstream channel end, that is parallel to and spaced from the first operative surface. The first operative surface may be a transparent material.

The flowcell also may have a second operative surface extending between the upstream channel end and the downstream channel end and configured to receive a second plurality of DNA templates, and a second reagent inlet fluidically connected to the upstream channel end at a location adjacent the second operative surface, wherein the buffer inlet is at a location spaced from the second operative surface. The first operative surface may be parallel to and facing the second operative surface. The first reagent inlet and the second reagent inlet may be fluidically connected or not fluidically connected upstream of the flowcell channel. The upstream channel end may have a predetermined height in a direction perpendicular the longitudinal axis, the first reagent inlet may have an exit portion adjacent to the upstream channel end that extends from a first point adjacent to the first operative surface to a second point that is spaced a first predetermined distance from the first operative surface in the direction perpendicular to the longitudinal axis, and the second reagent inlet may have an exit portion adjacent to the upstream channel end that extends from a third point adjacent to the second operative surface to a fourth point that is spaced a second predetermined distance from the second operative surface in the direction perpendicular to the longitudinal axis. The first predetermined distance may not equal or it may equal the second predetermined distance. The first predetermined distance and the second predetermined distance may equal to 0.4% to 50%, 1.4% to 18%, or 3.6% to 10.7% of the predetermined height. The predetermine height may be 140 micrometers, and the exit portions of the first and second reagent inlets may extend to points from their respective operative surfaces by distances of about 0.5 micrometers to about 50 micrometers about 2 micrometers to about 25 micrometers, or about 5 micrometers to about 15 micrometers.

In another exemplary aspect, there is provided a sequencing instrument having a flowcell as described above.

In another exemplary aspect, there is provided a method of operating a processing instrument, the method including providing a flowcell channel having an upstream channel end, a downstream channel end, a longitudinal axis extending from the upstream channel end to the downstream channel end, and a first operative surface extending between the upstream channel end and the downstream channel end and comprising a first plurality of DNA templates, providing a first reagent fluid to the upstream channel end channel at a first location adjacent the first operative surface, providing a barrier fluid that is different from the first reagent fluid to the upstream channel end at a second location spaced from the first operative surface, and passing the first reagent fluid and the barrier fluid through the flowcell channel under laminar flow conditions such that the first reagent fluid remains adjacent the first operative surface and the barrier fluid remains spaced from the first operative surface from the upstream channel end to the downstream channel end. The barrier fluid may remain spaced from the first operative surface from the upstream channel end to the downstream channel by a distance equal to 0.4% to 50%, 1.4% to 18%, or 3.6% to 10.7% of a total height of the flowcell channel at the upstream channel end as measured between the first operative surface and an opposite interior wall of the flowcell channel. The total height of the flowcell channel at the upstream channel end as measured between the first operative surface and an opposite interior wall of the flowcell channel may be about 140 micrometers, and the barrier fluid may remain spaced from the first operative surface from the upstream channel end to the downstream channel by a distance equal to about 0.5 micrometers to about 50 micrometers, about 2 micrometers to about 25 micrometers, or about 5 micrometers to about 15 micrometers. The first reagent fluid comprises may be provided in a direction parallel to the first operative surface. The barrier fluid may be provided in a direction parallel to the first operative surface.

The method also may include providing a second reagent fluid to the upstream channel end at a third location adjacent a second operative surface comprising a second plurality of DNA templates, wherein the second operative surface is spaced from the second location, and passing the second reagent fluid through the flowcell channel with the first reagent fluid and the barrier fluid under laminar flow conditions such that the second reagent fluid remains adjacent the second operative surface and the barrier fluid remains spaced from the second operative surface from the upstream channel end to the downstream channel end. The first operative surface may be parallel to and facing the second operative surface. The first reagent fluid and the second reagent fluid may be the same, or different. The second location may be spaced from the first operative surface by a first distance and the second location may be spaced from the second operative surface by a second distance, and the first distance may not equal or may equal the second distance. The first and second distance may be 0.4% to 50%, 1.4% to 18%, or 3.6% to 10.7% of a total height of the flowcell channel at the upstream channel end as measured between the first operative surface and an opposite interior wall of the flowcell channel. The total height of the flowcell channel may be about 140 micrometers, and the barrier fluid may remain spaced from each of the first and second operative surfaces by a distance equal to about 0.5 micrometers to about 50 micrometers, about 2 micrometers to about 25 micrometers, or about 5 micrometers to about 15 micrometers.

In another exemplary aspect, there is provided a method of operating a processing instrument, the method including providing a flowcell channel having an upstream channel end, a downstream channel end, a longitudinal axis extending from the upstream channel end to the downstream channel end, a first operative surface extending between the upstream channel end and the downstream channel end and comprising a first plurality of DNA templates, and a second operative surface extending between the upstream channel end and the downstream channel end and comprising a second plurality of DNA templates, providing a first reagent fluid comprising a first reactive chemistry to the upstream channel end channel at a first location adjacent the first operative surface, providing a second reagent fluid comprising a second reactive chemistry that is different from the first reactive chemistry to the upstream channel end at a second location adjacent the second operative surface, providing a barrier fluid that is different from the first reagent fluid and the second reagent fluid to the upstream channel end at a third location spaced from the first operative surface and the second operative surface, and between the first location and the second location, and passing the first reagent fluid and the barrier fluid through the flowcell channel under laminar flow conditions such that the first reagent fluid remains adjacent the first operative surface, the second reagent fluid remains adjacent to the second operative surface, and the barrier fluid remains spaced from the first operative surface and the second operative surface from the upstream channel end to the downstream channel end. The method may also include periodically passing the second reagent fluid through flowcell channel at the first location and the first reagent fluid through the flowcell at the second location, simultaneously with passing the barrier fluid through the flowcell channel at the third location, under laminar flow conditions such that the second reagent fluid remains adjacent the first operative surface, the first reagent fluid remains adjacent to the second operative surface, and the barrier fluid remains spaced from the first operative surface and the second operative surface from the upstream channel end to the downstream channel end. The method also may include imaging the first plurality of DNA templates when the first reagent fluid it passed through the flowcell channel at the first location, and imaging the second plurality of DNA templates when the first reagent fluid is passed through the flowcell at the second location.

In another exemplary aspect, there is provided a processing instrument programmed to perform the methods described above.

Other alternatives will be apparent to persons of ordinary skill in the art in view of the present disclosure.

The recitation of this summary of the invention is not intended to limit the claims of this or any related or unrelated application. Other aspects, embodiments, modifications to and features of the claimed invention will be apparent to persons of ordinary skill in view of the disclosures herein.

### BRIEF DESCRIPTION OF THE DRAWINGS

A better understanding of the exemplary embodiments may be had by reference to the attached drawings, in which like reference numbers designate like parts. The drawings are exemplary and not intended to limit the claims.

FIG. 1 is a schematic view of an exemplary embodiment of a sequencing instrument incorporating a flow cell.

FIGS. 2A and 2B are schematic cross-section elevation views of two permutations of an exemplary embodiment of a flow cell.

FIG. 3 is an exploded view of an exemplary embodiment of a flowcell.

FIG. 4 is a cross-section elevation view of the embodiment of FIG. 3.

FIG. 5 is a schematic cross-section elevation view of another exemplary embodiment of a flowcell.

FIG. 6 is an exploded view of an exemplary embodiment of a flowcell.

FIG. 7 is a plan view of an exemplary embodiment of a flowcell.

FIG. 8 is a cross-section view along line VIII-VIII of FIG. 7.

FIGS. 9A-9C are cross-section views of a an exemplary embodiment of a flowcell, shown in three different operative states.

### DETAILED DESCRIPTION

The inventors have determined that typical processing instrument flowcells can operate under conditions that waste a significant proportion of the expensive reactive chemicals that are necessary to perform the sequencing and imaging processes. In sequencing instruments, for example, these reactive chemicals may include an imaging chemistry comprising a buffer used during the imaging process, extension chemistry comprising fluorescently-labeled nucleotides that are used to extend the DNA templates, and a cleaving chemistry that is used to remove the fluorescent labels from the added nucleotides and remove a termination group to allow subsequent addition of nucleotides. Such reagents are expensive, and account for a large part of the operating costs of sequencing instruments. The exemplary embodiments described herein are provided in the context of DNA sequencing instruments, it will be readily appreciated that embodiments may be used in other kinds of processing instruments that use flowcells.

A typical microfluidic flowcell has very small dimensions (e.g., less than 1 mm in height), and has a relatively high surface area-to-volume ratio. As noted above, the height of the flowcell often is optimized between minimal reagent consumption, which urges the design towards reducing the flowcell height, and providing a reasonable pressure drop across the flowcell, which urges against reducing the flowcell height.

It has been determined that the fluid behavior in typical microfluidic flowcells is defined mainly by the fluid's interactions with the channel surfaces where viscous forces dominate over inertial forces. This leads to laminar flow conditions inside the channel, in which the reagent fluid (and the reactive chemicals located in the reagent fluid) moves in layers that maintain their relationship with one another throughout their passage through the channel (i.e., fluid at the top of the channel generally remains at the top, and fluid at the bottom generally remains at the bottom). For example, in a system having a flowcell channel with a height of 0.14 mm and a width of 6 mm, and a reagent fluid having approximately the same kinematic viscosity of water at 20° C. (˜1 mm²/s) flowing at 67 μL/s, the Reynolds number for the flow is approximately 22, which indicates that the flow is laminar.

Furthermore, it has been determined that the reactive chemicals in the reagent fluid can take considerable time to diffuse between the layers, even when the flow of reagent fluid has stopped. For example, fluorescently-labeled nucleotides having a diffusion coefficient of ˜200 μm²/s can take an average of about one minute to diffuse a 140 μm distance from the bottom of a flow cell to the top of a flow cell (and vice-versa).

As a result of these laminar flow and slow diffusion conditions, it is expected that a large proportion of the reactive chemicals may never be used. For example, in the exemplary flowcell system described above (i.e., a height of 0.14 mm, etc.), each sequencing cycle may include sequentially flooding the flowcell channel with a series of reactive chemicals, following each with a respective incubation time. The reactive chemicals located in layers furthest from the immobilized DNA templates are likely to never be used because they remain in the distant layer during the flooding process, and do not have sufficient time to diffuse to react with the DNA templates before the next sequencing step begins (the sequencing time potentially could be increased to increase the likelihood of reaction, but this sacrifices processing speed). In this diffusion-limited environment, a large proportion of reactive chemicals may be discarded in each reagent flooding cycle.

In view of the foregoing, it has been determined that the fluidic layers closest to the surfaces holding the DNA templates are the most relevant layers of the flow, because these layers are the first ones to bring reagents necessary for the biochemical reactions to the DNA templates, and because reactive chemicals beyond the closest layers are unlikely to diffuse into range to react with the DNA templates.

To address the problem of waste caused by laminar flow and the slow reaction kinetics of slowly-diffusing reactive chemicals, the inventors have provided a new flowcell design in which a supplemental barrier fluid flow is introduced adjacent to the reagent fluid to restrict the reagent fluid to a region proximal to the operative surface upon which the DNA templates are immobilized. Descriptions of exemplary embodiments follow, but it will be appreciated that the scope of the invention is not limited to any particular example, and the examples may be combined and modified in various ways, as will be understood by one of ordinary skill in the art in view of the present disclosure.

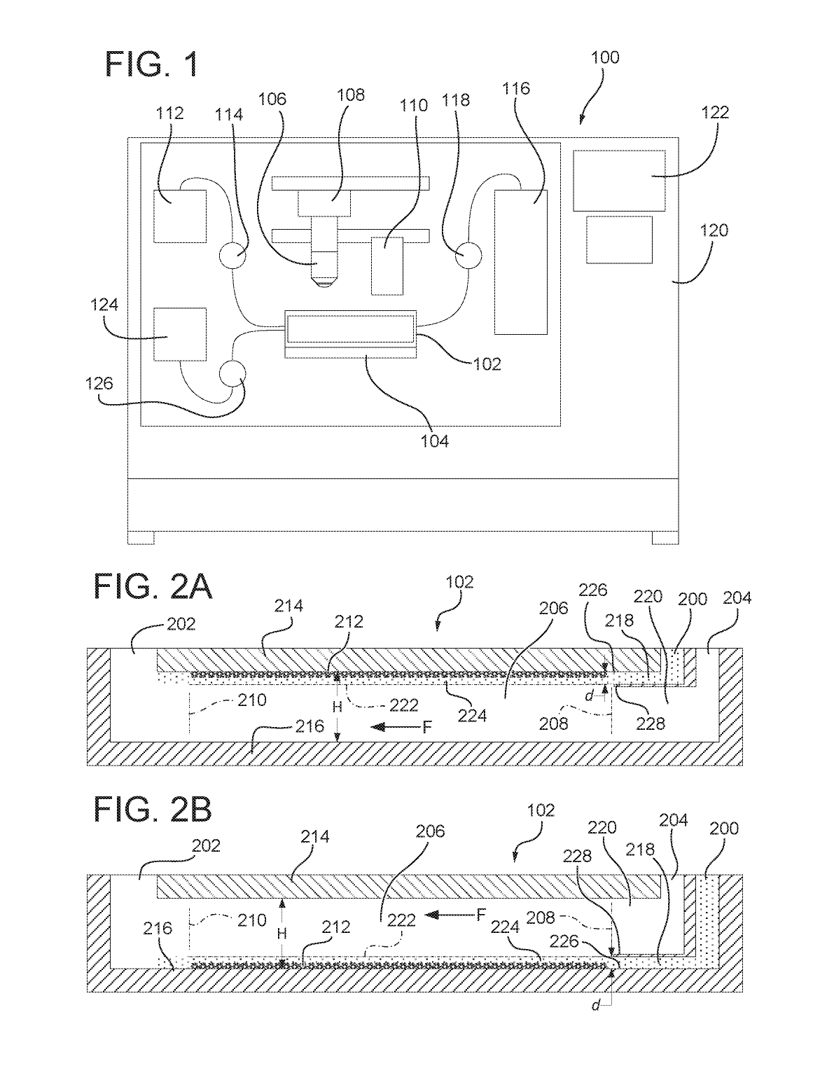

A first exemplary embodiment is illustrated in FIGS. 1, 2A and 2B. FIG. 1 shows a sequencing instrument 100 that incorporates a flowcell 102. The flowcell 102 may be permanently mounted in the instrument 100, or removable therefrom. The flowcell 102 may be mounted on a stage 104, which may be movable and may include a heating element (e.g., a so-called “Peltier” device or the like) that is used to control the temperature within the flowcell 102. The flowcell 102 is positioned adjacent an imaging device 106, such as a microscopic camera or the like, which may be mounted on a movable mount 108. A transport mechanism 110 (e.g., a robotic arm) also may be provided to manipulate or move the flowcell 102. The instrument 100 also includes one or more reagent containers 112 that are fluidically connected to one or more reagent inlets 200 into the flowcell 102. One or more pumps 114 may be fluidically connected between the reagent containers 112 and the flowcell inlet 200, and configured to pump the reagents into the flowcell 102 under positive pressure. The instrument 100 also includes one or more reagent recapture or waste receptacles 116 that are fluidically connected to a flowcell outlet 202. One or more pumps 118 may be fluidically connected between the waste receptacle 116 and the flowcell outlet 202 and configured to remove reagents from the flowcell 102. It is not expected that pumps 114, 118 will be required or used on both the inlet and outlet sides of the flowcell 102 in all embodiments, and either of the two types of pumps 114, 118 (i.e., positive-pressure or negative-pressure), or combinations of the same, may be used in various embodiments. The foregoing features may be integrated into a housing 120 having an operation control unit 122 to control automated operation of the parts. Reagent containers, waste receptacles, pumps, movable stages, heating devices, transport mechanisms, imaging devices, housings, and automated control systems are generally conventional in the art, and need no further explanation or discussion herein.

The instrument 100 further includes a barrier fluid supply 122 that is fluidically connected to a barrier fluid inlet 204 to the flowcell 102. A barrier fluid pump 124 also may be provided in the fluid path between the barrier fluid supply 122 and the secondary flowcell inlet 204, and configured to convey the barrier fluid to the flowcell 102 under positive pressure. Alternatively, the barrier fluid may be conveyed under negative pressure by a pump downstream of the flowcell 102.

FIGS. 2A and 2B show details of two exemplary embodiments of the flowcell 102. In each case, the flowcell 102 has a reagent inlet 200, an outlet 202, and a barrier fluid inlet 204. A flowcell channel 206 located inside the flowcell 102 extends from the reagent inlet 200 and barrier fluid inlet 204 at an upstream flowcell channel end 208, to the outlet 202 at a downstream flowcell channel end 210. The channel 206 may be straight, curved, have multiple parallel passages, or any other configuration suitable for the DNA sequencing process. In one example, the channel 206 comprises a straight passage with a rectangular cross-sectional shape having a channel height H of about 0.14 mm, and a width (i.e., the direction perpendicular to the height and the fluid flow direction F) of about 6 mm. The channel 206 preferably forms an enclosed passageway between the upstream flowcell channel end 208 and the downstream flowcell channel end 210, but additional inlets or outlets may be provided at points along the length of the channel 206, if desired. Other shapes and sizes may be used in other embodiments.

The channel 206 includes at least one operative surface upon which a plurality of DNA templates 212 are immobilized. Suitable structures for the operative surface and the manners in which DNA templates can be immobilized thereto are known in the art, and need not be described herein. Typically, the operative surfaces will be flat, so as to present the DNA templates in a single plane to facilitate imaging. In FIG. 2A, the DNA templates 212 are immobilized on the upper surface of the channel 206, which may comprise the lower surface of a transparent (e.g., borosilicate glass, polycarbonate, etc.) cover 214. In another example, shown in FIG. 2B, the DNA templates 212 are immobilized on the lower surface of the channel 206, which may comprise the upper surface of a flowcell base 216. The base 216 may be a transparent material, or a non-transparent material such as titanium-silicon alloys or other metals, ceramic, plastic, or the like.

The reagent inlet 200 is configured to direct its flow of reagent fluid into contact with the operative surface (i.e., the upper surface in FIG. 2A, and the lower surface in FIG. 2B). For example, the reagent inlet 200 may comprise an exit portion 218 that is parallel to and coincident with the height of the operative surface. Similarly, the barrier fluid inlet 204 is configured to direct its flow of barrier fluid at a location that is spaced from the operative surface. For example, the barrier fluid inlet 204 may have an exit portion 220 that is parallel to and spaced from the operative surface. In relation to the height H of the channel 206, the exit portion 218 of the reagent inlet 202 is positioned between the operative surface and the exit portion 220 of the barrier fluid inlet 204.

The proportional sizes of the exit portions 218, 220 may be selected as desired. For example, the exit portion 218 of the reagent inlet 202 may extend from a proximal location 226 that is located at the level of the operative surface, to a distal location 228 that is located at a distance d from the operative surface. For example, in a flowcell 102 having a channel height H of 140 μm at the upstream end, the distance d may be from 0.5 μm to 50 μm, from 2 μm to 25 μm, or most preferably from 5 μm to 15 μm. As another example, this distance d may be equal to about 0.4% to 36%, 1.4% to 18%, or 3.6% to 10.7% of the total channel height H as measured at the upstream end.

In use, reagent fluid including the reactive chemistry is directed through the reagent inlet 200 at the same time that barrier fluid is directed through the barrier fluid inlet 204. Operating under laminar flow conditions such as those described above, the barrier fluid abuts the reagent fluid at a boundary layer 222. A small amount of mixing may occur at the boundary layer 222, but for the most part the two fluid flows are expected to remain separate as they pass through the chamber 206 from the chamber inlet 208 to the chamber outlet 210. Thus, the barrier fluid effectively restricts the reagent fluid to a boundary region 224 adjacent the operative surface.

The position of the boundary layer 222 may be modified to adjust the size of the boundary region 224. It is expected that the boundary layer 222 will begin at or near the same distance d as the distal region 228 of the exit portion 218 of the reagent inlet 202. Assuming similar flow velocities for the reagent fluid and the barrier fluid, it is also expected that the boundary layer 222 will be generally parallel to the operative surface. For example, in a flowcell 102 having a channel height H of 140 μm, the boundary layer 222 may be positioned a distance from the operative surface of 0.5 μm to 50 μm, from 2 μm to 25 μm, or most preferably from 5 μm to 15 μm (or as another example, this boundary layer may be at a distance equal to about 0.4% to 50%, 1.4% to 18%, or 3.6% to 10.7% of the total channel height H). Some variation and degradation of the boundary layer 222 may occur, particularly at greater distances from the upstream end 208 of the channel 206. Where the boundary layer 222 is not entirely parallel to the operative surface, the measurement of the distance from the surface will be understood to be the distance at the upstream flow channel end 208. Degradation of the boundary layer 222 may be accounted for and the amount of or effects of such degradation reduced by modifying the starting position of the boundary layer 222, the length of the channel 206 in the flow direction F, the flow rates and pressures of the two fluids, and so on. Such modifications will be within the ability of a person of ordinary skill in the art, in view if this disclosure, without undue experimentation. Preferably, the boundary layer 222 remains intact for the full length of the operative surface that includes DNA templates, but this is not strictly required.

It is expected that a flowcell that uses a barrier fluid to restrict the reagent fluid to a boundary region 224 adjacent the operative surface will provide several benefits. For example, the barrier fluid effectively replaces a portion of the reagent fluid that would normally be used in the flowcell 102, which reduces reagent fluid consumption. This can provide a significant cost benefit by replacing a large portion of the reagent fluid with relatively inexpensive barrier fluid. Furthermore, the concentration of reactive chemicals in the reagent fluid can be increased in order to provide faster kinetics of diffusion-limited biological reactions, while still obtaining a cost reduction as compared to conventional systems. For example, the barrier fluid may replace 75% of the reagent fluid, and the concentration of reactive chemicals in the reagent fluid can be doubled, to obtain a 50% reduction in reactive chemical costs and nearly a 50% reduction in fluid chemistry costs (assuming the barrier fluid has a negligible cost as compared to the reactive chemicals). While similar reductions in costs might be obtained by simply reducing the size of the channel 206, doing so is expected to result in a significantly higher pressure drop across the flowcell 102, which may not be practical and may result in excessive clogging. The use of the barrier fluid allows the flowcell 102 to operate at conventional pressure differentials (and even reduced pressure differentials) while still obtaining the benefit of a concentrated flow of reagent fluid.

Any suitable barrier fluid may be used. For example, the barrier fluid may comprise an inexpensive and inert chemical, such as saccharide or a derivative thereof. The barrier fluid also may comprise a viscous fluid, such as polyethylene glycol, to decrease the diffusion of the reactive chemicals across the boundary layer 222 and out of the reagent flow. The barrier fluid also may comprise an immiscible fluid, such as mineral oil or silicone oil, to help prevent any exchange of reactive chemicals across the boundary layer 222. The buffer fluid also may comprise a non-reactive composition—i.e., a composition that does not chemically react as part of the sequencing process. Other alternatives will be apparent to persons of ordinary skill in the art in view of the present disclosure.

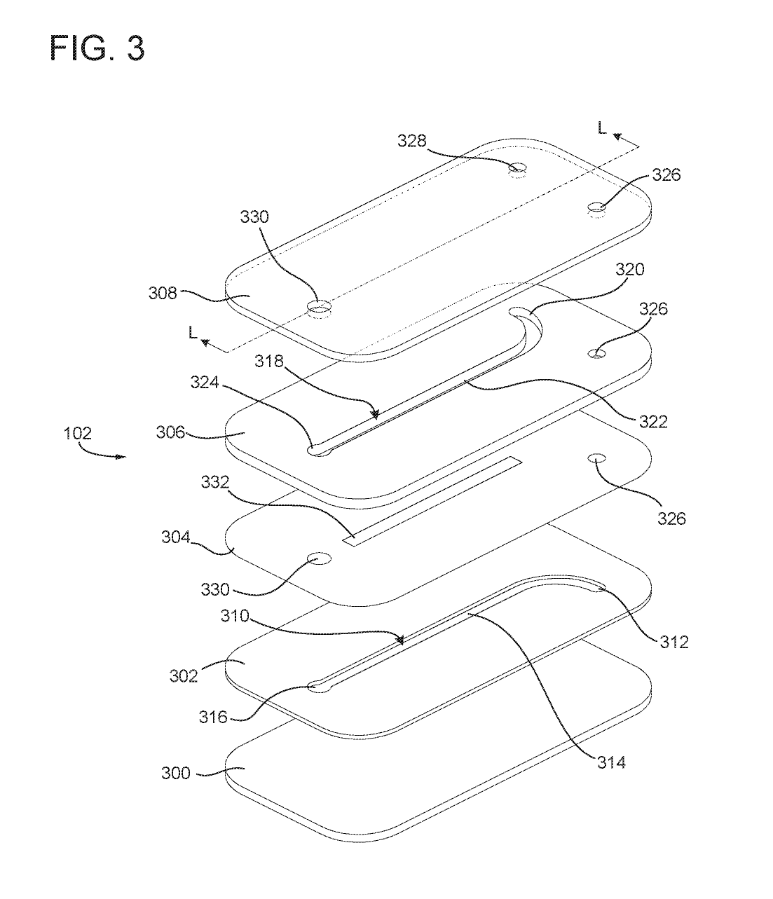

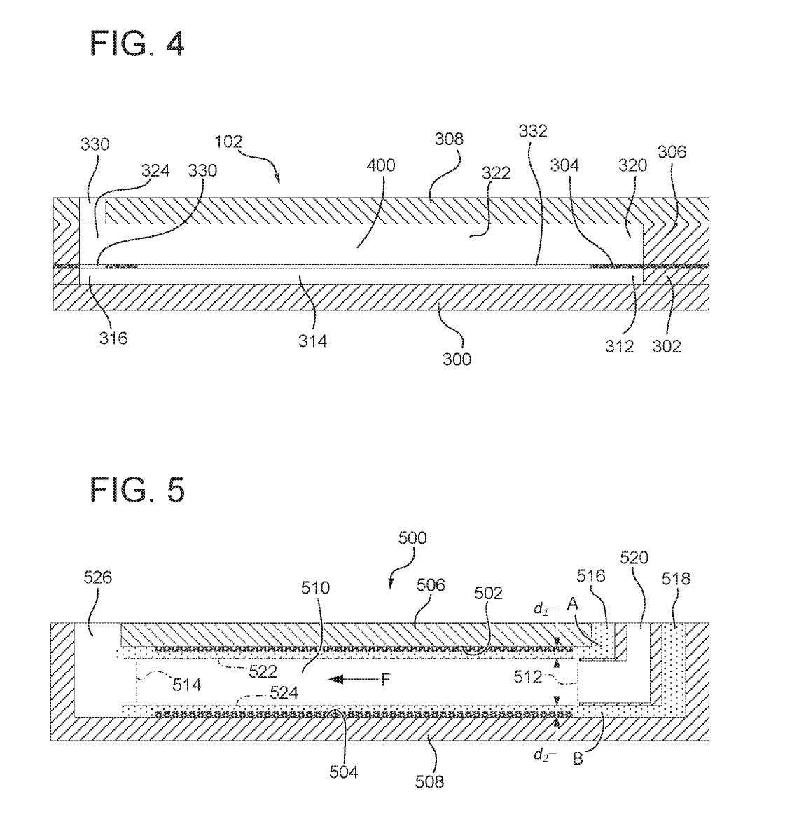

FIGS. 3 and 4 illustrate the construction of an exemplary flowcell 102. FIG. 3 shows the flowcell 102 in exploded view, and FIG. 4 shows a cutaway side view of the flowcell as viewed along the longitudinal centerline L-L of the flowcell 102. The flowcell 102 may be constructed of multiple layers that are laminated together using adhesives, welding, or any other suitable bonding technique. At the bottom, the flowcell 102 has a solid base layer 300. A first channel layer 302 is positioned on top of the base layer 300. A divider layer 304 is positioned on top of the first channel layer 302. A second channel layer 306 is positioned on top of the divider layer 304. Finally, a cover layer 308 is positioned on top of the second channel layer 306.

The first channel layer 302 includes a first channel 310 having a first end 312, a middle region 314, and a second end 316. The second channel layer 306 includes a second channel 318 having a first end 320, a middle region 322, and a second end 324. A series of first reagent openings 326 are provided in the layers above the first channel layer 302, and aligned with one another to provide a fluid passage through the cover 308 and to the first end 312 of the first channel 310. Similarly, one or more barrier fluid openings 328 are provided in the layers above the second channel layer 306, and aligned with one another to provide a fluid passage through the cover 308 and to the first end 320 of the second channel 318. A series of outlet openings 330 are provided in the cover 308 and divider layer 304, and aligned with one another to form a fluid passage through the cover 308 and to the second end 316 of the first channel 310, and the second end 324 of the second channel 318. Finally, the divider layer 304 includes a channel opening 332 that is aligned with the middle regions 314, 322 of the channels 310, 318, to combine and form a flowcell channel 400 that passes through the flowcell 102.

When assembled, the various parts and openings form the passages through the flowcell 102. A flow of reagent fluid can enter the reagent opening 326 through the cover 308, flow along the flowcell channel 400, and exit through the outlet openings 330. Similarly, a flow of barrier fluid can enter the barrier fluid opening 328 through the cover 308, flow along flowcell channel 400, and exit through the outlet openings 330. It will be appreciated that the arrangement of the openings and channels can be modified in any suitable way. For example, one or more of the reagent openings 326, the barrier fluid openings 328, or the outlet openings 330 may be configured to pass through the base 300, rather than the cover 308. The various openings 326, 328, 330 also may pass through the sides of the flowcell. Any configuration in which one or more openings are provided to convey fluid into and out of the first channel 310 and the second channel 318 may be used in other embodiments. Other alternatives will be apparent to persons of ordinary skill in the art in view of the present disclosure.

While not strictly necessary, the divider 304 provides a gasket to seal the first channel layer 302 to the second channel layer 306. Additional gasket layers (not shown) may be provided between the base layer 300 and the first channel layer 302, and the second channel layer 306 and the cover 308. The divider 304 also may be configured to divide the first channel 310 from the second channel 318 until they reach the flowcell channel 400, as shown in FIG. 4. For example, the divider opening 332 may begin at the terminus of the curved first ends 312, 320 of the first and second channels 310, 318. This may be helpful to ensure that the two fluids are traveling in the same direction at the time they contact one another, to prevent any possible turbulence that might cause unwanted mixing. The divider layer 304 also may be integrated into one of the channel layers 302, 304. For example, the second channel layer 306 may be machined such that the first end of the second channel 318 does not pass all the way through the second channel layer 306, while the middle region 322 does (instead of having the entire second channel 318 pass through the second channel layer 306 as shown in FIG. 3). Thus, the barrier fluid would not be able to contact the reagent fluid until they both reach the flowcell channel 400.

The foregoing construction is expected to provide suitable results and allow relatively simple manufacturing techniques. For example, the base layer 300, first channel layer 302 and second channel layer 314 may be made from sheet metal using conventional machining processes or from plastic using conventional molding and machining processes. The divider 306 may be integral with one of the other layers, constructed as a separate part in the same way as the other layers, or made of a pliable material (e.g., plastic film or sheet) that is formed using a stamping process. All embodiments are not intended to be limited to these construction techniques and materials, however, and other embodiments may use different constructions. For example, the flowcell 102 may be made of metal or plastic using machining, injection molding or three-dimensional printing to form the passages instead of stacking layers to form the passages. Other alternatives will be apparent to persons of ordinary skill in the art in view of the present disclosure.

It is also expected that the foregoing benefits of using a barrier fluid may be obtained in flowcells having multiple operative surfaces. For example, FIG. 5 shows an alternative flowcell 500 having a first operative surface 502 and a second operative surface 504. Each operative surface has a plurality of DNA templates immobilized thereon. In this case, the first operative surface 502 is formed on an cover 506 of the flowcell, and the second operative surface 504 is formed on the base 508 of the flowcell 500, but different surfaces may be used. The exemplary operative surfaces 502, 504 are provided on opposite sides of a channel 510 that passes through the flowcell 500. The channel 510 extends from an upstream end 512 to a downstream end 514, and may have any suitable shape and dimensions.

A first reagent inlet 516, a second reagent inlet 518 and a barrier fluid inlet 520 are fluidically connected to the upstream end 512 of the channel 510. The downstream end 514 of the channel 510 is fluidically connected to a flowcell outlet 526. As in the embodiment of FIG. 2, the inlets may have exit portions that extend parallel to the channel's flow direction F to help orient the incoming fluids into a laminar flow. The exit of the first reagent inlet 516 extends from a point adjacent to the first operative surface 502 to a point located a first distance d₁ from the first operative surface 502. Similarly, the exit of the second reagent inlet 518 extends from a point adjacent to the second operative surface 504 to a point located a first distance d₂ from the first operative surface 502. The distances may be like those described above (i.e., 0.5 μm to 50 μm, 2 μm to 25 μm, or 5 μm to 15 μm, or about 0.4% to 50%, 1.4% to 18%, or 3.6% to 10.7% of the total channel height), or have different dimensions. The d₁ dimension may equal the d₂ dimension, or different from the d₂ dimension. The exit of the barrier fluid inlet 520 is positioned between the exits of the first and second reagent inlets 516, 518.

As with the earlier embodiments, the first and second reagent inlets 516, 518 are fluidically connected to one or more reagent supplies, and may be have their own independent supply pumps or operate through a common pump. The barrier fluid inlet 520 is fluidically connected to a barrier fluid supply. Pumps are provided to convey reagents and barrier fluid into the flowcell under positive or negative pressure (or a combination of both).

This embodiment is expected to operate in the same manner as the earlier-described embodiments. In particular, reagent fluids may be directed through the first and second reagent inlets 516, 518 simultaneously with a flow of barrier fluid through the barrier fluid inlet 520. The fluids establish a laminar flow through the channel 510, and the barrier fluid creates first boundary layer 522 at the border of the first reagent flow, and a second boundary layer 524 at the border of the second reagent flow, with little or no mixing or diffusion across either boundary layer 522, 524.

This arrangement provides the benefits described above, such as reducing the consumption of expensive reactive chemical. This arrangement also allows the first and second operative surfaces 502, 504 to be subjected to two different reagent fluids. For example, the first operative surface 502 may be subjected to a first reagent flow A, and the second operative surface 504 may be subjected to a second reagent flow B that is different from the first reagent flow A. The first and second reagent flows A, B may comprise any chemistry used during the sequencing and imaging process. For example, one reagent flow may comprise chemistry used to cleave blockers from existing DNA templates to prepare the DNA templates to assimilate additional nucleotides during the extension process, and the other reagent flow may comprise fluorescently-labeled nucleotides that join to the DNA templates to perform the extension process. Other alternatives will be apparent to persons of ordinary skill in the art in view of the present disclosure. In use, the reagent flows can be alternatively applied to each operative surface 502, 504 in any desired sequence of operation, such as simultaneous, alternating, staggered, etc.

This ability to simultaneously expose the first and second operative surfaces 502, 504 to different chemistries can be used to simultaneously perform imaging on the DNA templates on one operative surface, while performing nucleobase extension on the DNA templates on the other operative surface. This may significantly increase the throughput rate of the instrument by reducing the lag time between subsequent imaging operations. To facilitate this, both the cover 506 and the base 508 may comprise a transparent surface, so that imaging can be performed from both sides of the flowcell 500. Furthermore, the barrier fluid may be opaque to provide a dark background during each imaging session, or have other properties helpful to the sequencing process. Other alternatives and advantages will be apparent to persons of ordinary skill in the art in view of the present disclosure.

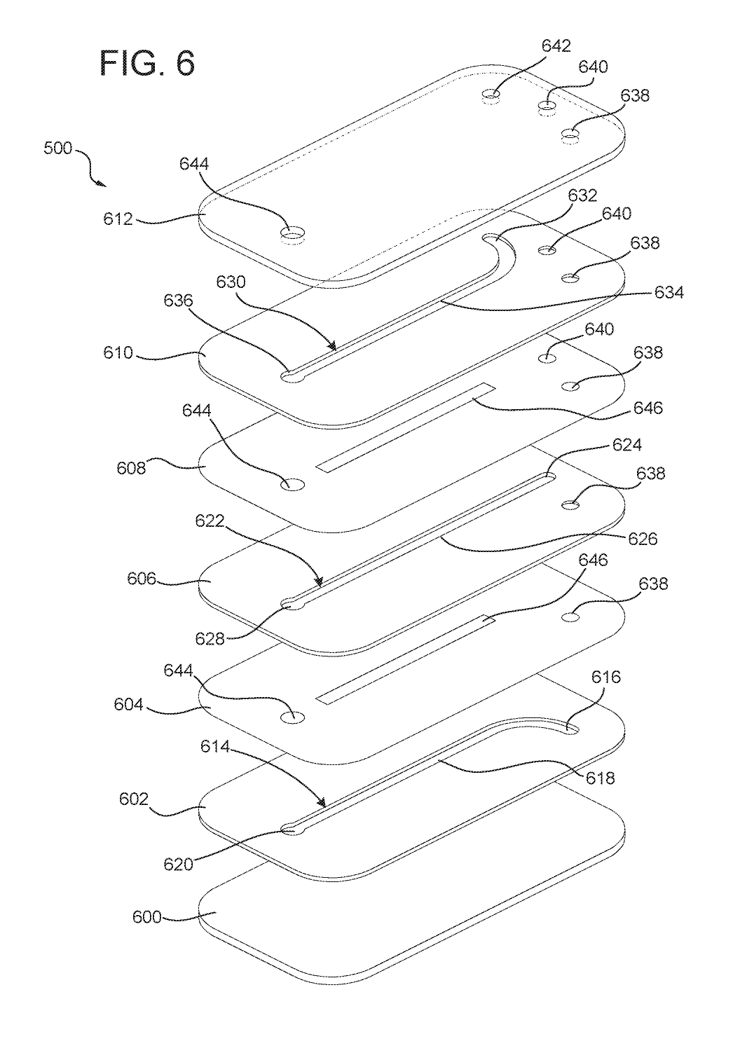

FIG. 6 illustrates an exemplary construction for a flowcell 500 configured with two operative surfaces and associated laminar flows. The flowcell 500 may be constructed of multiple layers that are laminated together using adhesives, welding, or any other suitable bonding technique. At the bottom, the flowcell 500 has a base layer 600, which may be partially or wholly transparent. A portion of the upper surface of the base layer 600 provides a first operative surface. A first channel layer 602 is positioned on top of the base layer 600. A first divider layer 604 is positioned on top of the first channel layer 602. A second channel layer 606 is positioned on top of the first divider layer 604. A second divider layer 608 is positioned on top of the second channel layer 606. A third channel layer 610 is positioned on top of the second divider layer 608. Finally, a cover 612, which may be partially or wholly transparent, is positioned on top of the third channel layer 610. A portion of the bottom surface of the cover 612 provides a second operative surface.

The first channel layer 602 includes a first channel 614 having a first end 616, a middle region 618, and a second end 620. The second channel layer 606 includes a second channel 622 having a first end 624, a middle region 626, and a second end 628. The third channel layer 610 includes a third channel 630 having a first end 632, a middle region 634, and a second end 636.

A series of first reagent openings 638 are provided in the layers above the first channel layer 602, and aligned with one another to provide a fluid passage through the cover 612 and to the first end 616 of the first channel 614. Similarly, a series of barrier fluid openings 640 are provided in the layers above the second channel layer 606, and aligned with one another to provide a fluid passage through the cover 612 and to the first end 624 of the second channel 622. A second reagent opening 642 is provided in the layer or layers above the third channel layer 610, and aligned to provide a fluid passage through the cover 612 and to the first end 632 of the third channel 630. A series of outlet openings 644 are provided in the divider layers 604, 608 and the cover 612, and aligned with one another to form a fluid passage through the cover 612 and to the second end 620 of the first channel 614, the second end 628 of the second channel 622, and the second end 636 of the third channel 630. Finally, the first and second divider layers 604, 608 include channel openings 646 that are aligned with the middle regions 618, 626, 634 of the channels 614, 622, 630, and combine to form a flowcell channel that passes through the flowcell 500. As with the embodiment of FIGS. 3 and 4, the channel openings 646 may be shaped to prevent contact between the incoming fluid flows before a desired point along the lengths of the channels 614, 622, 630.

When assembled, the various parts and openings form the necessary passages through the flowcell 500. This construction is expected to provide suitable results and allow relatively simple manufacturing techniques, as discussed in relation to the embodiment of FIGS. 3 and 4. Also as in those embodiments, the divider layers 604, 608 are not strictly required, and may be replaced, integrated into other layers, or modified as desired. Also, the various openings may be provided in any location so long as there is at least one opening to supply each of the channels with a respective fluid, and at least one opening to remove fluid from the channels. It is also anticipated that the first ends 616, 632 of the first and third channels 614, 630 may be connected to a common inlet to operate from a single source of reagent fluid. (In the shown embodiment, this may be accomplished by simply flipping over the third channel layer 610 about its long axis, which will fluidically connect the first and third channels 614, 630 upstream of the flowcell channel, and simultaneously feed all of the reagent passing through opening 638 in the cover 612 to both the first channel 614 and the third channel 630.) Other alternatives will be apparent to persons of ordinary skill in the art in view of the present disclosure.

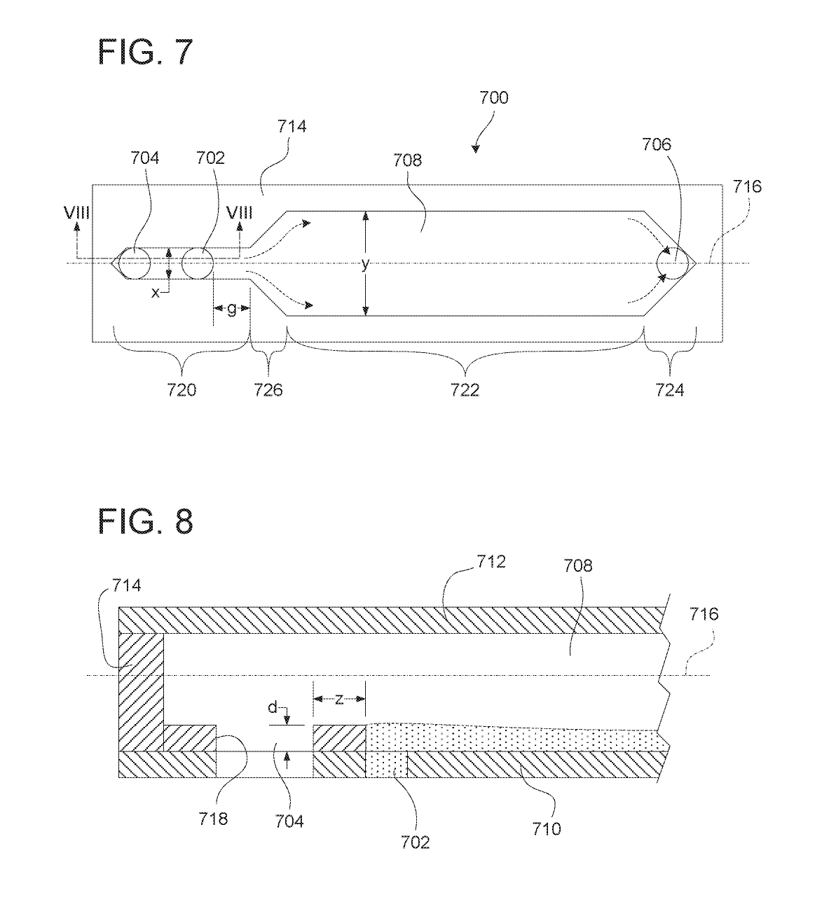

A further exemplary embodiment and its operation are illustrated in FIGS. 7-9C. This embodiment provides a flowcell 700 having a reagent inlet 702 and a barrier fluid inlet 704 and an outlet 706. A flowcell channel 708 extends from the reagent inlet 702 and barrier fluid inlet 704 at an upstream end of the channel 708, to the outlet 706 at a downstream end of the channel 708. The flowcell channel 708 is defined between a base 710 on the bottom, and a cover 712 on the top, one or both of which may be transparent. The base 710 and cover are joined and sealed at their perimeter by a perimeter spacer 714 that defines the perimeter shape of the channel 708.

The reagent inlet 702 and barrier fluid inlet 704 are provided as openings through the flowcell base 710 and/or cover 712. The outlet 706 may be an opening through the base 710 or cover 712. One or more of the reagent inlet 702, barrier fluid inlet 704 and outlet 706 alternatively may be formed as a passage through the perimeter spacer 714.

The reagent inlet 702 and barrier fluid inlet 704 may be spaced along a longitudinal axis 716 of the channel 708 by a distance z, with the reagent inlet 702 preferably (but not necessarily) being downstream of the barrier fluid inlet 704. The use of spaced inlets 702, 704 is expected to provide relatively straightforward manufacturing using existing production technologies for conventional flowcells. The distance z may be selected to optimize the development of suitable laminar flow conditions. The distance z also may be selected to accommodate constraints of the instrument, such as the requirement to provide fittings to the inlets 702, 704 and the like. In exemplary embodiments, the distance z may be 1-2 millimeters, but other distances may be used.

An inlet spacer 718 may be provided around the barrier fluid inlet 704 to space the barrier fluid inlet 704 relative to the reagent inlet 702 in a direction perpendicular to the longitudinal axis 716 by a predetermined distance d. The inlet spacer 718 may be integral to or separate from the perimeter spacer 714. The inlet spacer 718 also may be movable to alter the distance d. For example, the inlet spacer 718 may comprise a tube that is movable through the opening that defines the barrier fluid inlet 704 to place the end of the barrier fluid inlet 704 at alternative distances d from the reagent inlet 702. Alternatively, the inlet spacer 718 may be removable and replaceable with an inlet spacer 718 of a different thickness to define a new distance d.

The inlet spacer 718 is expected to guide the inert barrier fluid over the reagent fluid in a way that reduces or eliminates uncontrolled effects at the meeting zone between the fluids that might impair the development of the desired laminar flow conditions. For example, a Venturi effect may occur when the barrier fluid passes over the reagent inlet 702, in which the moving reagent fluid exerts additional low pressure to the reagent inlet fluid and pulls portions of the reagent fluid into the barrier fluid. This can lead to microfluidic droplets of reagent fluid forming within the barrier fluid. Localized mixing at the junction of the fluids also may occur due to other fluid mechanics. The inlet spacer 718 may reduce or eliminate this and other phenomena, and embodiments are not intended to be bound to any particular theory of operation.

As shown in an exemplary manner in the plan view of FIG. 7, the channel 708 may expand in width downstream of the inlets 702, 704. The use of an expanded flowcell channel 708 is expected to make the channel width independent of the fluid inlet opening size, while helping to minimize reagent usage. For example, the channel 708 may begin with an inlet zone 720 at the reagent inlet 702 and barrier fluid inlet 704 which has a first width x (e.g., 1 mm), expand laterally in an intermediate zone 722 (which preferably contains DNA templates) to a second width y, and then contract in the lateral direction in an exit zone 724 that surrounds the outlet 706. The channel 708 may include an upstream transition region 726 between the inlet zone 720 and the intermediate zone 722, in which the perimeter wall of the perimeter spacer 714 expands gradually from the first width x to the second width y. The perimeter wall may be straight (such as shown) or curved in any suitable manner to help promote the maintenance of laminar flow. Similarly, the exit zone 724 may be formed by the perimeter wall of the perimeter spacer 714, and have any suitable straight or curved profile.

The first width x preferably is equal to the width of the reagent inlet 702. This is expected to ensure that the incoming reagent distributes across the full width of the intermediate zone 722 of the channel 708. In contrast, if the first width x is greater than the reagent inlet 702 width, it is expected that laminar flow effects may prevent the reagent from expanding laterally to the full width y of the intermediate zone 722. Nevertheless, in embodiments in which it is not necessary for the reagent to extend across the full width y of the intermediate zone 722, the first width x may be greater than the width of the inlet channel 702. The second width y may be any width (e.g. 6 mm), but preferably is selected such that the fluids maintain a sufficient degree of laminar flow and separation throughout the channel 708.

It is expected that, when the reagent and barrier fluid are pumped through their respective inlets 702, 704, the fluids will meet at the reagent inlet 702 and create an overlapping multiphase laminar flow over the whole channel width x within the inlet zone 720. When the channel 708 expands to the intermediate zone 722, overlapping laminar flow will expand accordingly due to laminar behavior of the fluids. In some embodiments, it may be necessary or desirable to provide a certain distance between the reagent inlet 702 and the end of the inlet zone 720 (i.e., before lateral expansion) to stabilize the laminar behavior and reduce or eliminate fluidic effects that might disturb the balanced expansion of the two fluids when they reach the intermediate zone 722. For example a gap distance g in the range of 0.2 to 5.0 mm or even longer may be provided between the reagent inlet 702 and the end of the inlet zone 720.

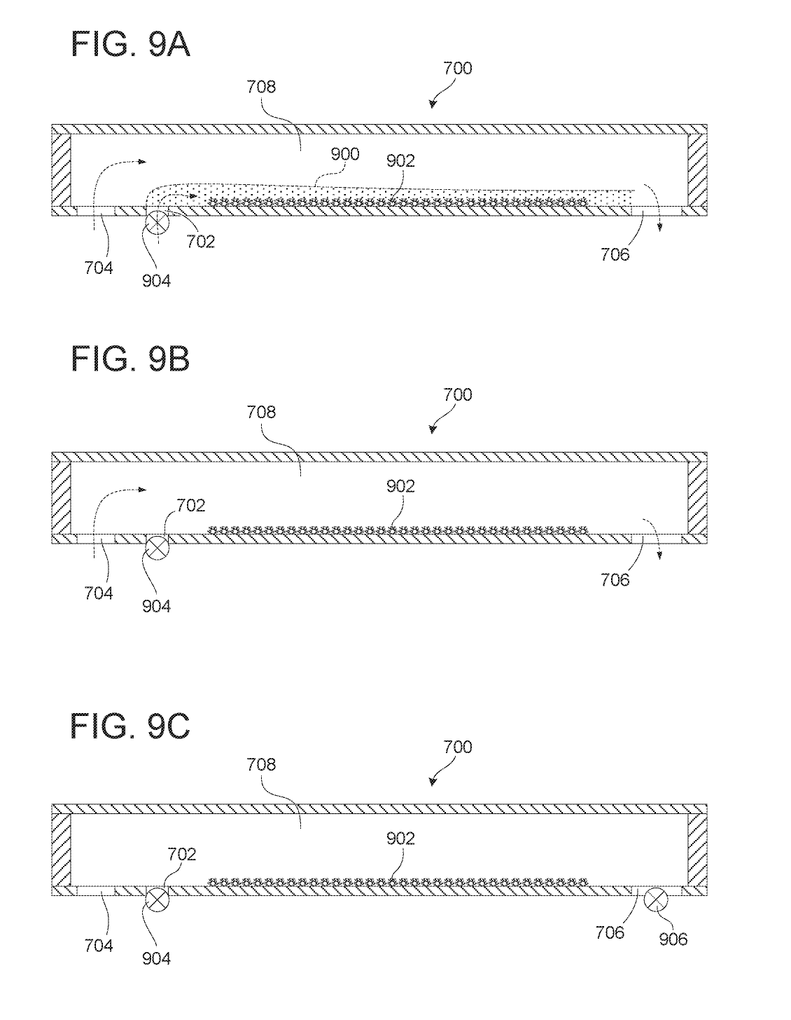

Examples of how the foregoing flowcell 700 (or similar flowcells) may be used are described and illustrated with reference to FIGS. 9A-C. FIGS. 9A-C show the flowcell 700 without an inlet spacer surrounding the barrier fluid inlet 704, but an inlet spacer may be provided if desired.

FIG. 9A shows the flowcell 700 in a first operative state, in which reagent fluid is passing through the reagent inlet 702 and barrier fluid is passing through the barrier fluid inlet 704. As explained previously, a boundary layer 900 forms between the reagent and the barrier fluid, which holds the reagent close to a layer of DNA templates 902 located on the flowcell base 710. The reagent and barrier fluid exit the flowcell channel 708 at the outlet 706. In this “reaction mode” of operation, the DNA templates are exposed to the reagent to perform any desirable reaction (e.g., nucleobase extension). The reagent and barrier fluid may be pumped through the channel 708 using any suitable mechanism, such as applying negative pressure downstream of the flowcell 700 to pull them through the channel 708, or applying positive pressure upstream of the flowcell 700 to push them through the channel 708. A combination of methods also may be used. For example, a suction pump may be provided downstream of the flowcell 700, and one or both of the reagent and the barrier fluid may also be conveyed by a pump or pumps upstream of the flowcell 700.

One or more of the reagent inlet 702, barrier fluid inlet 704 and outlet 706 may include a flow valve to control the passage of fluid. Such valves may be located anywhere along the flow passage leading to the respective opening, but it may be preferable for the valve to be as close as possible to the opening to minimize residual flow when the valve is closed. For example, the reagent inlet 702 may have a reagent inlet valve 904 that, when opened, allows reagent to pass into the channel 708 when a downstream pump is activated to pull the barrier fluid into the channel 708. The reagent valve 904 preferably may be gradually adjusted to vary the flow resistance and thus flow rate of the reagent passing through the reagent inlet 702. This feature may be used, for example, to gradually adjust and selectively choose the volume proportions of the reagent and the barrier fluid.

A second operation mode is illustrated in FIG. 9B. In this mode, the reagent inlet valve 904 is closed so that only inert barrier fluid passes through the flowcell channel 708. This mode may be used to provide a complete replacement (i.e., a “wash”) of reactive chemistry from the reagent, which can be helpful to perform multiple different reactions without mixing the reagents, and so on.

A third operation mode is illustrated in FIG. 9C. Here, an outlet valve 906 has been closed to prevent any fluid from passing through the outlet 706. Assuming the fluid is normally pumped through the flowcell channel 708 by a pump located downstream of the outlet 706, closing the outlet valve 906 eliminates any pressure difference between the reagent inlet 702 and the barrier fluid inlet 704, and no flow passes through the flowcell channel 708. If the system includes one or more pumps located upstream of the flowcell 700, it may be necessary to also turn of such pumps to prevent backflow of reagent into the barrier fluid inlet 704, or vice versa, or a reagent valve 904 or a valve at the barrier fluid inlet 704 may be closed to prevent such backflow. Other alternatives will be apparent to persons of ordinary skill in the art in view of the present disclosure.

It will be appreciated that the foregoing embodiments may be modified in various ways. For example, the barrier fluid inlet 704 may be downstream of the reagent inlet 702, or the flowcell channel 708 may have a third inlet to receive a second flow of reagent fluid to provide reactive chemistry to the top and bottom of the channel 708. Also, the feature of an expanding-width flowcell channel also may be provided in the earlier embodiments describe herein in relation to FIGS. 1-6. Other alternatives will be apparent to persons of ordinary skill in the art in view of the present disclosure.

The present disclosure describes a number of new, useful and nonobvious features and/or combinations of features that may be used alone or together. It is expected that embodiments may be particularly helpful to reduce the cost of goods associated with high-throughput nucleic acid sequencing systems, but other benefits may be provided, and it will be appreciated that reduced cost is not necessarily required in all embodiments. While the embodiments described herein have generally been explained in the context of sequencing by syntheses processes, it will be appreciated that embodiments may be configured for use in other sequencing processes that use visual observation of chemical labels. The embodiments described herein are all exemplary, and are not intended to limit the scope of the inventions. It will be appreciated that the inventions described herein can be modified and adapted in various and equivalent ways, and all such modifications and adaptations are intended to be included in the scope of this disclosure and the appended claims.

We claim:

1. A flowcell for a processing instrument, the flowcell comprising:
a flowcell channel extending along a longitudinal axis from an upstream channel end to a downstream channel end, and having a first operative surface extending between the upstream channel end and the downstream channel end, the first operative surface being configured to receive a first plurality of DNA templates;
a first reagent inlet fluidically connected to the upstream channel end at a first location adjacent the first operative surface;
a buffer inlet fluidically connected to the upstream channel end at a second location spaced from the first operative surface;
an outlet fluidically connected to the downstream channel end.

2. The flowcell of claim 1, wherein:
the upstream channel end has a predetermined height in a direction perpendicular to the longitudinal axis;
the first reagent inlet comprises an exit portion adjacent to the upstream channel end, and the exit portion extends from a first point adjacent to the first operative surface to a second point that is spaced a predetermined distance from the first operative surface in the direction perpendicular the longitudinal axis; and
the predetermined distance is equal to 0.4% to 50% of the predetermined height.

3. The flowcell of claim 1, wherein:
the upstream channel end has a predetermined height in a direction perpendicular to the longitudinal axis;
the first reagent inlet comprises an exit portion adjacent to the upstream channel end, and the exit portion extends from a first point adjacent to the first operative surface to a second point that is spaced a predetermined distance from the first operative surface in the direction perpendicular the longitudinal axis; and
the predetermined distance is equal to 1.4% to 18% of the predetermined height.

4. The flowcell of claim 1, wherein:
the upstream channel end has a predetermined height in a direction perpendicular to the longitudinal axis;
the first reagent inlet comprises an exit portion adjacent to the upstream channel end, and the exit portion extends from a first point adjacent to the first operative surface to a second point that is spaced a predetermined distance from the first operative surface in the direction perpendicular the longitudinal axis; and
the predetermined distance is equal to 3.6% to 10.7% of the predetermined height.

5. The flowcell of claim 1, wherein:
the upstream channel end has a predetermined height of about 140 micrometers in a direction perpendicular the longitudinal axis;
the first reagent inlet comprises an exit portion adjacent to the upstream channel end, and the exit portion extends from a first point adjacent to the first operative surface to a second point that is spaced about 0.5 micrometers to about 50 micrometers from the first operative surface in the direction perpendicular the longitudinal axis.

6. The flowcell of claim 1, wherein:
the upstream channel end has a predetermined height of about 140 micrometers in a direction perpendicular the longitudinal axis;
the first reagent inlet comprises an exit portion adjacent to the upstream channel end, and the exit portion extends from a first point adjacent to the first operative surface to a second point that is spaced about 2 micrometers to about 25 micrometers from the first operative surface in the direction perpendicular the longitudinal axis.

7. The flowcell of claim 1, wherein:
the upstream channel end has a predetermined height of about 140 micrometers in a direction perpendicular the longitudinal axis;
the first reagent inlet comprises an exit portion adjacent to the upstream channel end, and the exit portion extends from a first point adjacent to the first operative surface to a second point that is spaced about 5 micrometers to about 15 micrometers from the first operative surface in the direction perpendicular the longitudinal axis.

8. The flowcell of claim 1, wherein:
the first reagent inlet comprises a first reagent inlet exit portion located immediately upstream of the upstream channel end, and the exit portion is parallel to and coincident with the first operative surface; and
the buffer inlet comprises a buffer inlet exit portion located immediately upstream of the upstream channel end, and the buffer inlet exit portion is parallel to and spaced from the first operative surface.

9. The flowcell of claim 1, wherein the first operative surface comprises a transparent material.

10. The flowcell of claim 1, wherein:
the flowcell channel further comprises a second operative surface extending between the upstream channel end and the downstream channel end and configured to receive a second plurality of DNA templates;
the flowcell further comprises a second reagent inlet fluidically connected to the upstream channel end at a third location adjacent the second operative surface; and
wherein the second location is spaced from the second operative surface.

11. The flowcell of claim 10, wherein the first operative surface is parallel to and facing the second operative surface.

12. The flowcell of claim 10, wherein the first reagent inlet and the second reagent inlet are fluidically connected at a location upstream of the flowcell channel.

13. A method of operating a processing instrument, the method comprising:
providing a flowcell channel extending along a longitudinal axis from an upstream channel end to a downstream channel end, and having a first operative surface extending between the upstream channel end and the downstream channel end, the first operative surface comprising a first plurality of DNA templates;
providing a first reagent fluid to the upstream channel end channel at a first location adjacent the first operative surface;
providing a barrier fluid that is different from the first reagent fluid to the upstream channel end at a second location spaced from the first operative surface; and
passing the first reagent fluid and the barrier fluid through the flowcell channel under laminar flow conditions such that the first reagent fluid remains adjacent the first operative surface and the barrier fluid remains spaced from the first operative surface from the upstream channel end to the downstream channel end.

14. The method of claim 13, wherein the barrier fluid remains spaced from the first operative surface from the upstream channel end to the downstream channel end by a distance equal to 0.4% to 50% of a total height of the flowcell channel at the upstream channel end as measured between the first operative surface and an opposite interior wall of the flowcell channel.

15. The method of claim 13, wherein the barrier fluid remains spaced from the first operative surface from the upstream channel end to the downstream channel end by a distance equal to 1.4% to 18% of a total height of the flowcell channel at the upstream channel end as measured between the first operative surface and an opposite interior wall of the flowcell channel.

16. The method of claim 13, wherein the barrier fluid remains spaced from the first operative surface from the upstream channel end to the downstream channel end by a distance equal to 3.6% to 10.7% of a total height of the flowcell channel at the upstream channel end as measured between the first operative surface and an opposite interior wall of the flowcell channel.

17. The method of claim 13, wherein a total height of the flowcell channel at the upstream channel end as measured between the first operative surface and an opposite interior wall of the flowcell channel is about 140 micrometers, and the barrier fluid remains spaced from the first operative surface from the upstream channel end to the downstream channel end by a distance equal to about 0.5 micrometers to about 50 micrometers.

18. The method of claim 13, wherein a total height of the flowcell channel at the upstream channel end as measured between the first operative surface and an opposite interior wall of the flowcell channel is about 140 micrometers, and the barrier fluid remains spaced from the first operative surface from the upstream channel end to the downstream channel end by a distance equal to about 2 micrometers to about 25 micrometers.

19. The method of claim 13, wherein a total height of the flowcell channel at the upstream channel end as measured between the first operative surface and an opposite interior wall of the flowcell channel is about 140 micrometers, and the barrier fluid remains spaced from the first operative surface from the upstream channel end to the downstream channel end by a distance equal to about 5 micrometers to about 15 micrometers.

20. The method of claim 13, wherein providing the first reagent fluid comprises providing the first reagent fluid in a direction parallel to the first operative surface.

21. The method of claim 13, wherein providing a barrier fluid comprises providing the barrier fluid in a direction parallel to the first operative surface.

22. The method of claim 13, further comprising:
providing a second reagent fluid to the upstream channel end at a third location adjacent a second operative surface comprising a second plurality of DNA templates, wherein the second operative surface is spaced from the second location; and
passing the second reagent fluid through the flowcell channel with the first reagent fluid, the second reagent fluid and the barrier fluid under laminar flow conditions, such that the second reagent fluid remains adjacent the second operative surface and the barrier fluid remains spaced from the second operative surface from the upstream channel end to the downstream channel end.

23. The method of claim 22, wherein the first operative surface is parallel to and facing the second operative surface.

24. The method of claim 22, wherein the first reagent fluid and the second reagent fluid are the same.

25. A method of operating a processing instrument, the method comprising:
providing a flowcell channel having an upstream channel end, a downstream channel end, a longitudinal axis extending from the upstream channel end to the downstream channel end, a first operative surface extending between the upstream channel end and the downstream channel end and comprising a first plurality of DNA templates, and a second operative surface extending between the upstream channel end and the downstream channel end and comprising a second plurality of DNA templates;
providing a first reagent fluid comprising a first reactive chemistry to the upstream channel end channel at a first location adjacent the first operative surface;
providing a second reagent fluid comprising a second reactive chemistry that is different from the first reactive chemistry to the upstream channel end at a second location adjacent the second operative surface;
providing a barrier fluid that is different from the first reagent fluid and the second reagent fluid to the upstream channel end at a third location spaced from the first operative surface and the second operative surface, and between the first location and the second location; and
passing the first reagent fluid and the barrier fluid through the flowcell channel under laminar flow conditions such that the first reagent fluid remains adjacent the first operative surface, the second reagent fluid remains adjacent to the second operative surface, and the barrier fluid remains spaced from the first operative surface and the second operative surface from the upstream channel end to the downstream channel end.

26. The method of claim 25, further comprising periodically passing the second reagent fluid through flowcell channel at the first location and the first reagent fluid through the flowcell at the second location, simultaneously with passing the barrier fluid through the flowcell channel at the third location, under laminar flow conditions such that the second reagent fluid remains adjacent the first operative surface, the first reagent fluid remains adjacent to the second operative surface, and the barrier fluid remains spaced from the first operative surface and the second operative surface from the upstream channel end to the downstream channel end.

27. The method of claim 26, further comprising imaging the first plurality of DNA templates when the first reagent fluid it passed through the flowcell channel at the first location, and imaging the second plurality of DNA templates when the first reagent fluid is passed through the flowcell at the second location.
